(19) United States
(12) Patent Application Publication
(10) Pub. No.: US 2010/0313503 A1
(43) Pub. Date: Dec. 16, 2010

(54) Multifunctional Floor Pods

(75) Inventor: John R. Mangiardi, Maur (Zurich) (CH)

Correspondence Address: DECHERT LLP, P.O. BOX 390460, MOUNTAIN VIEW, CA, 94039-0460, US

(73) Assignee: Optimus Services, LLC, Greenwich, CT (US)

(21) Appl. No.: 12/779,516

(22) Filed: May 13, 2010

Related U.S. Application Data
(63) Continuation of application No. 11/996,038, filed on Feb. 6, 2008, now abandoned.
(60) Provisional application No. 60/701,106, filed on Jul. 20, 2005.

(51) Int. Cl. E04C 2/52; B62B 5/00

(52) U.S. Cl. 52/220.1; 280/29

(57) ABSTRACT

A floor pod (100) for indoor use, including a vertically movable bollard adapted to present utilities and provide security function, presented in a watertight housing (110) is disclosed.

### CROSS-REFERENCE TO RELATED APPLICATIONS

This application claims the benefit of provisional patent application U.S. Ser. No. 60/701,106, filed Jul. 20, 2005 by the present inventor. The contents of U.S. Ser. No. 60/701,106 are expressly incorporated herein by reference thereto.

The following references are hereby explicitly incorporated by reference thereto:
U.S. Pat. No. 5,462,384
U.S. Pat. No. 5,787,879
U.S. Pat. No. 7,052,201
Applications filed along with present application by current inventor on this date entitled:
   IN-CEILING FOCUS LOCATED SURGICAL LIGHTING
   HOSPITAL OPERATING ROOM RE-DESIGN
   AMBIENT LIGHTING IN HOSPITAL SURGICAL ENVIRONMENTS
   USE OF ULTRAVIOLET GERMICIDAL IRRADIATION IN HEALTH CARE ENVIRONMENTS
   IN-WALL WASTE RECEPTACLES FOR HOSPITAL AND LABORATORY ENVIRONMENTS
   RE-DESIGN OF OPERATING ROOM TABLES
   ROBOTIC FLOOR CLEANING WITH STERILE, DISPOSABLE CARTRIDGES

### BACKGROUND OF THE INVENTION

1. Field of Invention

The present invention relates to devices and methods for providing utility connections and optionally security means within the home, hospital, laboratory, or other in-door environment.

2. Background of the Invention

The present invention is a multifunctional floor pod similar in some respects to outdoor dispensing bollards. Dispensing bollards are a derivative of known outdoor safety and security bollards. Located in areas where traffic control is a concern the latter type of bollard provides an obstruction to the flow of vehicular traffic. The capability of retracting into an underground chamber thereby allows traffic to pass over the bollards. Retraction and distention of the out-door safety bollards is known to use manual, mechanical means for displacement of the bollard. In U.S. Pat. No. 5,462,384 of Arlandis, a self-contained dispensing bollard of variable height is described. The device therein attempts to provide a bollard body adapted to dispense utility connections such as for outdoor vendors.

An indoor-bollard would have to be adapted for indoor use. Particularly, the bollard should include the already known function of retractability. However, previous out-door bollards, such as those described by Arlandis, have a top-surface which does not provide a flush surface with the surrounding ground when the bollard is in its retracted position. This makes cleaning of the top-surface more difficult. Further, the top “anchor” is unsightly. In-door markets, such as grocery stores or retail stores, seek to provide the customer with a shopping experience in which the operations of the store (such as the dispensing of utilities) are concealed or masked. Further, a top anchor or bulge from a dispensing bollard is a safety risk in that persons can easily trip over the bollard's bulge.

In addition, an indoor-bollard should be able to retract and distend under powered means. Preferably, the bollard would also allow vertical displacement by a control distant from the actual bollard, thereby allowing, for example, a store manager the ability to retract all unused store bollards at day's end, to allow cleaning of the top surfaces, or for other purposes such as allowing goods to be transported past.

Certain indoor environments would requite even further modifications of existing designs to be functionally usable. A floor-pod in a hospital or laboratory environment would be of great benefit because of space and safety concerns. Further, multiple floor-pods in such environments could eliminate the amount of floor-space covered by wiring or other connections (a safety hazard because of the chance a person will trip over cabling or be exposed to a frayed cable). A user would have multiple locations to choose an optimal utility source point, i.e. an optimally positioned floor pod, when the floor pods are located strategically about the working environment.

However, standard dispensing bollards would not be adapted for use in such environments because of safety concerns. The same is applicable, although to a lesser extent, for use of standard dispensing bollards in retail or home use. While a flush surface would allow cleaning of the top portion of a bollard, accidental spillage of waste on or about standard bollards would result in contamination of the bollards in a manner that is not practical to clean and sterilize. An invention that can provide a floor pod that resists intrusion of contaminants into the floor pod interior and the floor pod cavity, whether in the retracted or distended position would be of benefit. Also, a floor pod with specialized connections or receptacles for utilities, or even a docking port for devices would also be of benefit. For example, a surgical cart could be adapted to dock with a distended floor pod eliminating the safety hazard of cables altogether as no cabled connection whatsoever is required in a docking configuration.

Lastly, a floor pod that can function as a security device—in-doors—would be of benefit, particularly for those in retail where a floor pod could help control foot traffic and even prevent intrusions of vehicles and equipment into the store environment. Also, heavy equipment located in a storage facility can be secured therein, using floor pods to prevent their removal.

### OBJECTS AND SUMMARY OF THE INVENTION

It is an object of this invention to provide a floor pod capable of dispensing utilities such as electricity, water, gasses, and capable of providing connections to data ports such as Ethernet, serial, parallel, USB (universal serial bus), and Bluetooth (and other wireless).

A device which allows electrical power, fluids, gases, and links to data lines such as Ethernet, USB, and the like to a user in an indoor setting such as a residential home, an office building, a hospital operating room, a university laboratory, a retail store, a restaurant, or convention center is therefore described. The device also eliminates extraneous cables, wires, and hoses from the floor or elsewhere.

It is another object of this invention to provide a floor pod capable of retraction and distention by vertical displacement, thereby providing a flush surface when retracted, and said displacement occurring by powered means and controlled at the pod or by a wired or wireless control distant from the pod.

The floor pod, consistent with the present invention, provides powered means such as by a piston and support block combination which when powered by an electro-pneumatic, electro-hydraulic, or preferably a electro-mechanical motor/generator retracts or distends the bollard-like pod. The actuator for controlling the vertical displacement of the pod may be controlled by a wired or wireless connection to a point distant from the pod, such as in a control box resting on a wall, or optionally on the pod itself. The controls would not only provide actuation of the displacement of the pod but may also provide security means, such as a by an electronic lock-out device, to prevent unauthorized use of the utility connections.

The pod resides in a housing beneath a floor. Outside utilities are run under the floor to the pod, thereby providing a cable/wire/hose free environment above ground. When in the retracted state, the floor pod top surface, which is slightly larger than then the housing below, rests in a cavity adapted to fit the top surface in a manner resulting in a flush surface.

It is yet another object of this invention to provide a floor pod that is capable of providing security and safety by functioning as a control on foot or vehicular traffic. It is also designed to withstand the force and pressure of persons or equipment standing or rolling over the top of the floor pod.

The floor pod, consistent with the present invention, will anchor within a sub-flooring or anchor to the surface below a sub-flooring. The anchoring of the device will provide in part the structural integrity necessary for the floor pods functioning as a security/safety device. Further, the housing of the floor pod and the floor pod itself may be comprised of materials known to provide great tensile, compression, shear, and other translational force resistance such as steel, reinforced steel, or even lighter materials such as aluminum or magnesium used in thicknesses and amounts commonly known to provide such resistance. For example, the floor pod can be comprised of polished steel, anchored to and comprised within a housing in a concrete subflooring such that in its retracted state, a person weighing 100 kg or more, or a device weighing up to 300 kg may rest its weight for a prolonged period, such as an hour, on a surface area of about 80 square centimeters.

In its distended state, the bollard may be designed, by additional reinforcement, to prevent the intrusion of a vehicle into, for example, a store area. The bollard would be further adapted to provide a means for emergency shut-off of utilities to prevent creation of a hazard, such as by a force-sensor that operates to shut-off a distant control valve and/or power junction when said force-sensor realizes an impact. In areas where foot traffic needs to be controlled, an array of floor pods in their distended state can provide a clear signal that entry is prohibited. It is conceived that the floor pod may further utilize lights, sound, or other visual/auditory indicators as warning signals. The signals may warn against entry or that a component of the floor pod is malfunctioning.

It is another object of this invention to provide a floor pod that is adapted to prevent intrusion of contaminants into the interior of the pod or the cavity containing said pod.

Therefore, as an improvement over the prior art, the floor pod is not only retractable below ground level when the top surface must be cleaned or its use is not required but it provides a flush surface by retracting into its cavity, a seal on its perimeter making it watertight. A seal on its bottom perimeter (when the pod is distended) also provides a watertight seal when not retracted. These seals utilize at least one O-ring with at least one seal compressed in the floor pods retracted or distended state. For added protection, it is conceived that additional O-rings may be placed within the housing or along the cavity's perimeter. As such, the floor pod would be designed to withstand intense water, waste exposure such as by a spraying with a fire hose.

It is also an object of this invention to provide a floor pod that can integrate with other floor pods in an environment, thereby providing multiple access points for utility or other connections or for other purposes such as security.

It is another object of this invention to provide a floor pod that can allow docking of devices adapted to meet the receptacles or connections presented on the pod.

It is yet another object of this invention to provide a floor pod that can dispense a variety of utilities, particularly gases, using a single adaptor type for any receptacles or connections on said pod.

It is also an object of this invention to provide a method for using the various embodiments of the above described floor pods in indoor hospital or laboratory environment.

It is an object of this invention to provide a method for using the various embodiments of the above described floor pods in indoor residential, commercial, or industrial environments.

At least one of the above objects is met in whole or in part by the invention. Further objects are apparent by the following description and claims.

### BRIEF DESCRIPTION OF THE DRAWINGS

The present invention can best be understood in connection with the accompanying drawings. It is noted that the invention is not limited to the precise embodiments shown in drawings, in which:

FIG. 1 is a side cross-sectional view of a pod in the raised position;

FIG. 2 is a side cross-sectional view of the pod depicted in FIG. 1 in a lowered position;

FIG. 3 is a perspective view of the pod showing the side docking connections and the side receptacle connections;

FIG. 4 is a side view detail of docking operation between the pod top surface and bottom of an anesthesiology cart;

FIG. 5 is a side view detail of an alternative embodiment of the docking operation between the pod top surface and bottom of an anesthesiology cart;

FIG. 6 is a perspective view of an alternative embodiment of the pod showing the side docking connections and the side receptacle connections.

### BRIEF DESCRIPTION OF REFERENCE NUMERALS

100 Floor Pod; 102 Floor Pod Top Surface; 104 Floor Pod Side and Top Sleeve; 104′ Floor Pod Side and Bottom Sleeve; 106 Support Block; 108 Cylinder; 110 Floor Pod Internal Housing; 112 Piston; 114 Anchor; 116. Actuator; 118 sub-floor terminus and surface; 120 Floor; 122 Sub-floor; 132 Upper Lip Seal; 132′ Lower Seal Lip; 200 Receptacles; 200′ Wires, Cables, Hoses; 202 Universal Docking Receptacles; 204 Connector; 206 Universal Docking Connector; 208 Docking Connector; 210 Receptacles; 212 Convenience Receptacle; 214 Shutter; 220 Gas; 222 Water; 224 Electricity; 226 Adaptor/Switch Combination; 228 Adaptor/Switch Compartment; 230 Adaptor Compartment; 232 Wire/Hose/Cable; 234 Adaptor Connection; 236 Connector; 238 Control Box; 240 Switch/Multiplexer; 242 Adaptor Utility Path; 244 I/O Path for Control Box; 250 Cart

### DETAILED DESCRIPTION OF THE INVENTION

As a general matter, a typical embodiment of the invention comprises a pod with various utility connections. The pod will usually have a first utility panel, which dispenses non-gas utilities such as electricity or data lines. The pod will then also have a second panel, usually opposite the first panel, which dispenses gas utilities, such as oxygen, and/or provides scavenging adaptors, such as vacuum. The second panel of gas/scavenging utilities typically presents a single, universal adaptor type. The selection of a universal adaptor provides ease of use and rapid interchange of connections. The universal adaptor may be a male style, such as an Ohmeda style hose barb or a Schrader (swivel or non-swivel) style hose barb; or the female connect versions of the same hose barbs. Also contemplated are alternative universal adaptor types such as Ohmeda or Schrader style NPT (National Pipe Thread) fittings (both male and female). Chemetron and Puritan style universal adaptors are also usable. Schrader fittings are preferred.

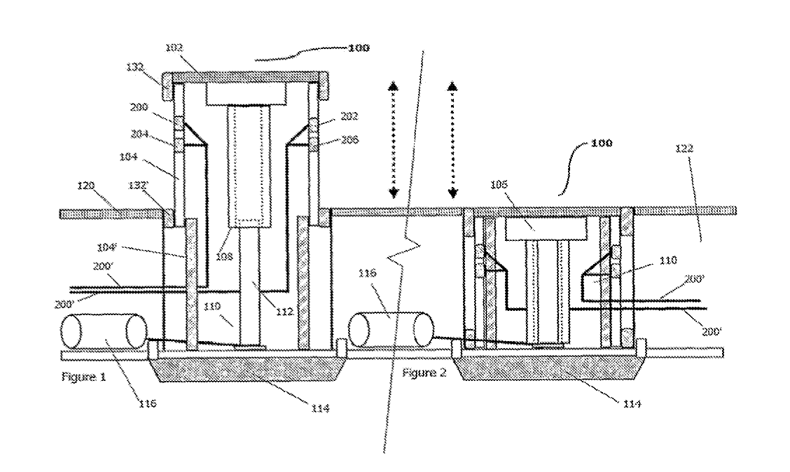

In a preferred embodiment, pods 100 are shown in cross-section in housings 110 in FIG. 1 (in a raised position) and FIG. 2 (in a lowered position). The pods 100 are placed directly into flooring 122 that may consist of any standard below floor materials such as concrete or stone. The pods are placed into cavities formed by, for example, wet-cutting areas for the pods and are anchored below the floor material 122 at 118 by floor pod anchor 114. A piston 112 within cylinder 108 and support block 106 are used for raising or lowering pod 100 via lifting forces, such as by fluid from either an electro-pneumatic, electro-hydraulic, or electro-mechanical generator 116. Pods 100 have an outer side housing 104 an inner housing 104′. Any outer side of pod 100 such as side housing 104 or the top surface 102 may be adapted to provide connections to utilities or means for docking with devices such as a specially designed surgical cart, thereby providing utilities to these or other devices—such as with 200, 202, 204, 206 provided by cable 200′, wire 200′, or hose 200′. The top surface 120 of the finished floor will be flush with top of pod 102 when lowered as seen in FIG. 2.

The outer, spool-like surface 104 with seals 132 and 132′ functions to further provide a watertight junction. Seal 132′, which may run along part of or along the entire length of side 104, provides a watertight seal when pod 100 is distended and seal 132 provides a watertight seal when pod 100 is retracted. The seals may be made of any known materials offering good wear and water resistance such as the materials used to manufacture Buna-N (Nitrile) O-Rings, which are copolymers of butadiene and acrylonitrile. They may also, in a preferred embodiment, be made of materials used to manufacture ethylene-propylene (EPDM, EPR, EP) O-rings, which are copolymers of ethylene and propylene, or terpolymers with butadiene. The latter offer superior resistance to water. Other materials commonly used to provide water-tightness and good wearability characteristics may also be used. Support column 106 provides additional structural integrity to the pod 100.

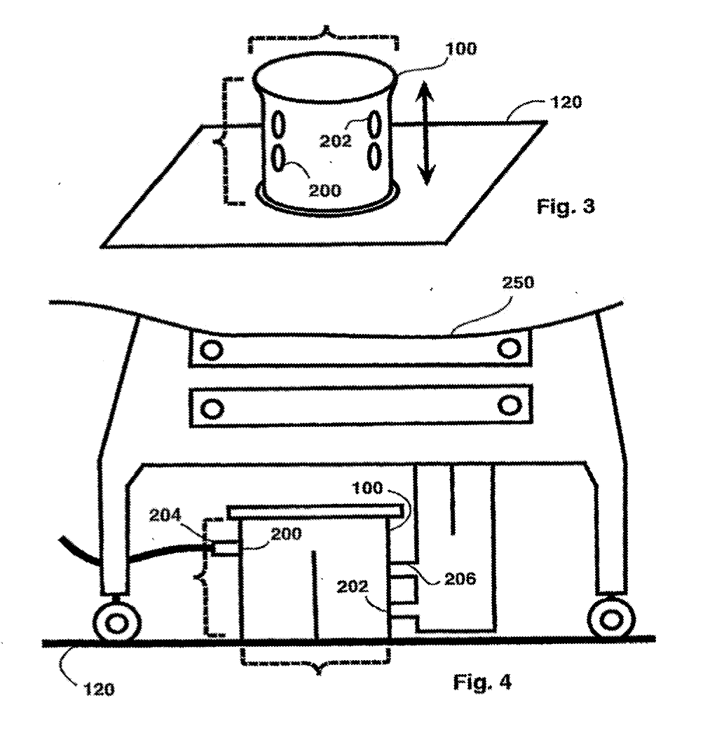

FIG. 3 is a perspective view of one preferred embodiment of the pod 100 showing a spool-like shape at the upper and bottom portions and the side docking connections 202 and the side receptacle connections 200. The pod is in its distended state above floor 120.

FIG. 4 shows the relationship between the first set of male connectors 206 on the side docking plate of cart 250 and the female receptacles 202 within the side of pod 100 during docking or retraction. Receptors 202 are specially designed for connection with a surgical cart adapted for docking. The relationship between the second set of female connectors 200 and an outside connection 204, such as to a device requiring electricity, is shown. The receptacles 200 are designed to be adapted to a variety of utilities. The connections may be reversed in an alternative embodiment in which male connectors 206 and/or 204 are female receptacles and female receptacles 202 and/or 200 are male connectors. Under floor 120, female receptacles 200 and 202 are connected to various utilities. In this embodiment, it is preferred that the male and female receptacles providing gases or scavenging lines are of a universal type, i.e. a single adaptor type will be used for all gas/vacuum connections.

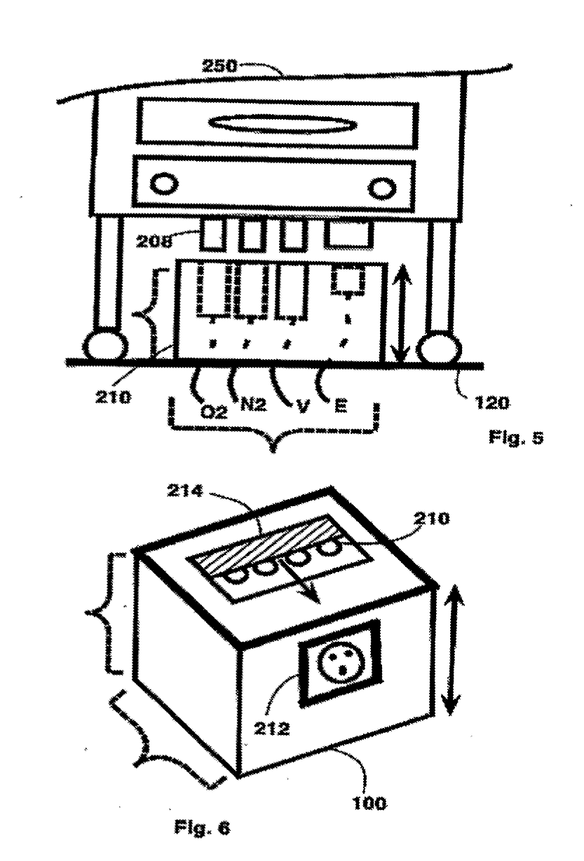

FIG. 5 shows an alternative embodiment of the pod. The relationship between the male connectors 208 on the bottom docking plate of cart 250 and the female receptacles 210 within the top end of pod 100 during docking or retraction are shown. The connections may be reversed in an alternative embodiment in which male connectors 208 are female receptacles and female receptacles 210 are male connectors. Under floor 120, female receptacles 210 are connected via hoses to oxygen, vacuum, nitrous oxide, and via cable to provide electrical power. In an alternate embodiment, the male and female receptacles may be of a universal type, i.e. a single adaptor will be used for all utility connections.

FIG. 6 shows a close-up of the top surface of the alternative embodiment of pod 100 shown in FIG. 5, showing a sealable compartment where female receptacles 210 are exposed by sliding of automatic door or shutter 214. When door 214 is closed, it seals watertight so that the top surface can be washed down. The top edge of pod 100 also seals against the floor 120 (not shown), thereby preventing water drainage down the sides of the device. Hospital-grade convenience outlet 212 is also provided for equipment that may be placed on raised pod 100 (besides a cart, e.g. 250, requiring utilities).

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention. It is further known that other modifications may be made to the present invention, without departing the scope of the invention, as noted in the appended claims.

1. An in-door multifunctional floor pod of variable height adapted to provide utilities, comprising:
a vertically movable bollard, watertight in its retracted or distended state, inserted in a housing in a floor, said bollard having an outer structure adapted to present utility connections, said outer structure having a top surface, said top surface of said outer structure forming a flush surface with said floor when said bollard is retracted, and an upper and lower sealing lip adapted to provide a watertight seal when said bollard is retracted or distended,
an inner structure adapted to receive utilities from connections running beneath a floor and having a powered actuator which vertically moves said bollard.

2. The floor pod of claim 1 in which said powered actuator is powered by a motor, an electro-pneumatic generator, an electro-mechanical generator, or an electro-hydraulic generator.

3. The floor pod of claim 1 in which said utility connections are provided by presentation of utility receptacles.

4. The floor pod of claim 1 in which said utility connections are provided by presentation of connectors.

5. The floor pod of claim 1 in which any of said utility connections are provided by presentation of a universal adaptor adapted to provide gases, vacuum, scavenging means, aspiration means, and other like utilities.

6. The floor pod of claim 1 in which said utility connections are provided by presentation of a dock comprising any combination of receptacles, connectors, and universal adaptors.

7. The floor pod of claim 1 in which said powered actuator is controlled by a wired or wireless control box adapted to allow a user to control the up/down position of said floor pod.

8. The floor pod of claim 1 in which said top surface is comprised of materials that are non-porous and sterilizable.

9. The floor pod of claim 1 in which the utilities provided include any combination of electricity, water, gasses, data ports such as Ethernet, serial, parallel, USB (universal serial bus), and Bluetooth (and other wireless), and scavenging connections such as an aspirating or vacuum connection.

10. The floor pod of claim 1 in which said bollard is docked to a device adapted to join with any said presented utilities by said device being placed on bollard or said device being rolled onto bollard.

11. An array of floor pods, comprising at least two floor pods as claimed in claim 1.

12. The array of claim 11 in which said powered actuators of said floor pods are controlled by a wired or wireless control box adapted to allow a user to control the up/down position of said floor pods.

13. A surgical cart adapted to dock with a floor pod as described in claim 1.

14. (canceled)
